(19) United States
(12) Patent Application Publication
(10) Pub. No.: US 2015/0104821 A1
(43) Pub. Date: Apr. 16, 2015

(54) Device and Method for Sorting and Labeling of Biological Systems based on the Ionic-Electric Dynamics

(71) Applicant: Stefan Goetz, Durham, NC (US)

(72) Inventors: Stefan Goetz, Durham, NC (US); Arthur Singer, Munich (DE); Thomas Weyh, Munich (DE); Florian Helling, Munich (DE)

(21) Appl. No.: 14/052,784

(22) Filed: Oct. 14, 2013

(51) Int. Cl. G01N 33/487; G06F 19/24; G01N 27/416; G06F 19/12

(52) U.S. Cl. CPC G01N 33/48728; G06F 19/12; G06F 19/24; G01N 27/416
USPC 435/29; 435/287.1; 435/288.7; 204/403.01; 205/777.5; 600/300

(57) ABSTRACT

The present invention relates to a method and a device for creating digital copies of the ionic-electric dynamics of cells or cell compartments, such as organisms, or an at least partly identifying dataset that allows sorting decisions in vitro and in vivo. Sorting of cells and cell compartments based on electrical cell behavior can be applied, for instance, to transgenic compartments, in order to, e.g., detect the successful expression of certain channel proteins in an industrial process, to screen for (side) effects of certain agents or substances on the ionic-electrical cell behavior and involved proteins. In that context, the invention can be used as an alternative to complex and/or time consuming patch-clamp screenings as well as for general quality control reasons.

### TECHNICAL FIELD

The present invention relates to a method and a device for creating digital copies of the ionic-electric dynamics of cells or cell compartments, such as organisms, or an at least partly identifying dataset that allows sorting decisions in vitro and in vivo. Sorting of cells and cell compartments based on electrical cell behavior can be applied, for instance, to transgenic compartments, in order to, e.g., detect the successful expression of certain channel proteins in an industrial process, to screen for (side) effects of certain agents or substances on the ionic-electrical cell behavior and involved proteins. In that context, the invention can be used as an alternative to complex and/or time consuming patch-clamp screenings as well as for general quality control reasons.

### BACKGROUND ART

Electrical behavior plays a major role in biological cells. A central part of intracellular mechanisms is controlled ionic-electrically. Furthermore a big part of communication between individual cells or cellular units works ionic-electrical (see B. Alberts. Molecular Biology of the Cell. Garland Science, 3rd Edition, New York, 1994). In neurons for instance, the ionic-electrical communication and signal transmission—within and across the cell—as well as the regulation of key effects such as synaptic plasticity, of parts of the metabolism or even the mitosis via ionic-electrical communication channels are well known. However, nearly all cell types genetically express a certain distinct ionic-electrical system. The ionic-electrical system as such subsumes all the time variant mechanisms which are sustained by ions and their modifications—i.e., their movements in space or electro-chemical responses—and which therefore are electrically detectable.

As a general rule, this ionic-electrical system in cells is operated by proteins. The most prominent among these are channel proteins and receptors in and at the membrane of a cell or cell organelles. In addition, there are also numerous microstructures, such as aquaporins, gap junctions or transporter-proteins. The special feature of these structures for this case is their strong dynamic characteristics. Many of these proteins gain their functions by a fine interplay of nano-mechanical and electrostatic/electrodynamic mechanisms, partly with chemical influence, which result in a nonlinear behavior with very characteristic properties. In particular, voltage sensitive elements, e.g. voltage controlled ion channels for sodium, calcium or potassium ions, show very characteristic activation and deactivation dynamics with very distinct nonlinearity and voltage-dependency.

The overall dynamic behavior of a certain mechanism relates to the interplay of many involved and characteristic elements. The absence or addition of a component, such as a certain channel protein, or a malfunction, e.g., due to a missing or not expressed channel sub unit, become clearly apparent in the dynamic behavior and can therefore be easily detected and characterized even in the resulting behavior of the whole system.

### INVENTION SUMMARY

In general the invention consists of a method and associated apparatus, aimed at analysis, detection, reproduction by means of mathematical models, classification and based on these classifications decision making, on/of/about characteristic stimulus-response-dynamics of an ionic-electrical system or mechanism within cells, cell aggregate or whole organisms.

Consequently, biological single cells, functionally or mechanically/physically contiguous cell aggregates and whole organisms composed of biological cells will be referred to by the term cells in the following. These can be examined using this invention in vivo, i.e., in an organism or in a part of an organism, as well as in vitro, i.e., outside of a biological organism, whereas a culture medium provides the physiological conditions for the cell's metabolism and/or growth and/or development artificially.

Proteins participating in such ionic-electrical cell mechanisms are of far-reaching technical importance. For example in the pharmaceutical industries and in biotechnology, cells or whole organisms are generated with distinct characteristics (often by means of genetical processes). A lot of these designed distinct characteristics particularly apply to one of the ionic-electrical mechanisms of cells. Thereby particular genes are often modified, transfected or merely genetically expressed. However, even on an industrial scale, these processes do not show a 100% success rate. Therefore a sorting and/or or quality control means is required following such design processes, wherein cells expressing the designed, distinct characteristics are biologically, chemically, pharmacologically or physically separated from the cells not showing the characteristics aimed for to make sure that all finally sold products have been successfully modified and therefore show the specific characteristics. The sorting or quality control means may identify a biological, chemical, pharmacological or physical marking, e.g., using colorants, immune-complex reactions, radioactive-labeling or in this context, other, scientifically known methods of marking. All mentioned or equivalent processes for marking, separation, sorting or any other identification based on characteristics of the ionic-electrical system are referred to as sorting in the following.

Classical techniques for this final sorting or quality control are relatively similar regarding cells or whole organisms. In the latter case, e.g., samples can be taken in order to apply cell-biology techniques. Typical techniques are fluorescence activated cell sorting (FACS), numerous assay techniques (also immuno-assays), electrophoresis, electro-chemical detection techniques, DNA sequencers, bio-sensors, chromatography or mass spectrometry.

The invention at hand relates to a procedure and the associated apparatus which characterizes, identifies and differentiates cells, cell aggregates or whole organisms based on the individual dynamic characteristics of ionic-electrical systems. In a distinct embodiment of this invention these are used for automated decision-making, particularly for sorting.

Moreover, the detected individual dynamic properties may also serve as a basis for other decisions that do not require any special sorting, e.g., to selectively treat only certain groups as selectively as possible with a particular biological, pharmacological or physical treatment.

Another field of application of the invention at hand is pharmacological, toxicological as well as chemical screening. In the (systematical) development and in approval of agents/drugs and toxins, their effects on a plurality of cell mechanisms are generally examined in a screening procedure. Thereby, the majority of analyses applies to elements of the ionic-electrical mechanisms, such as receptors and ion channels. Usually, these examinations are performed using well-known patch-clamp techniques. Therein single cells, typically from primary cultures or technically generated cell lines with manipulated and well-defined characteristics, are individually tested for abnormal behavior of the proteins of the ionic-electrical mechanisms. In a patch-clamp measurement, the electrical behavior of the cell is, for instance, detected by inserting a thin pipette into the cytosol (so called whole cell measurement) or by isolating a single/some membrane proteins (cell attached or single channel measurements). In this well-know experimental technique in electro-physiology an experienced experimenter is needed in general. Due to the manual execution, this technique is costly as well as time consuming in systematical screenings.

Although several technical approaches to automate the patch-clamp technique using polar patch-clamp chips (see invention N. Fertig et al.; DE19936302, EP1775586, US2007/0087327, US2005/0009171) and/or robots, these methods in general depend on particular cell characteristics (such as cell shape, the existence of individual cells outside of a cell aggregate, no adherent growth, or similar limiting conditions) and/or exclude primary cultures or cell aggregates. Moreover they do not solve the problem that proteins whose specific behavior has to be verified must be analyzed individually by functionally isolating them using, e.g., chemical/pharmacological blockers or other substances which manipulate—in the majority of cases inhibit or deactivate—biological mechanisms, particularly signal cascades.

It would be considerably cheaper to use automated so-called multi-electrode-arrays to observe and influence the ionic-electrical behavior of cultivated cells or cell aggregates. These arrays consist of a typical culture substrate, such as glass or plastic, with conductive electrodes on the surface. Parts of these electrodes may be electrically isolated in order achieve a local selectivity by prohibiting a local current flow to the overlying cell medium or cells and by using a multitude of electrodes. The technically complicated process of getting an electrical access to a cell in patch-clamp is omitted. This procedure enables simple automated cultivation of cells on appropriate, electrically conductive electrode structures. Hereby the electrodes can be used for the detection of ionic-electrical responses as well as for the excitation, in addition or instead of exciting them chemically, pharmacologically, biologically or physically (e.g., according to mechanism under study by adding certain substances, optically, mechanically, thermally, etc.). In both cases, the dynamics can be controlled very precisely. The transient shape of the electrical stimulus (current-, voltage- or load-controlled) can be adjusted very accurately over a wide spectrum (e.g., rectangular pulses, sinusoidal pulses or arbitrary intermediate forms as well as more complex signals similar to noise or so called random-walk patterns). Similar dynamical control of equivalent measures can be achieved with other physical or chemical stimulation methods, e.g., the light intensity and its spectrum for the stimulation of receptors, of separately expressed proteins or just of the cell membrane. The detection of the responses using appropriate multi-channel-amplifiers is state of the art.

Similar to the procedure described above, the individual dynamics of a ionic-electrical system can be characterized, identified and distinguished from a common or reference state of cells in order to, e.g., determine and quantify the (side) effects of a drug.

Moreover, it is not only possible to detect changes, but also their cause and the functional or spatial location of the change (e.g., a certain (channel) protein) can be identified or rather be calculated from the measurements.

Such screenings analyzing the overall dynamic behavior can be performed for whole organisms in vivo. The present invention analyzes the expression of distinct proteins or their artificial or natural mutations, epigenetical modifications or biochemical/physical alterations, e.g., by phosphorylation, by influences or modulation due to chemically or physically (usually electro-statically) attached or bound substances or molecules; among the latter are, for example, neuromodulators and messengers. This can be performed simultaneously on one or more neurons, whose stimulus-response-dynamics are being analyzed. Similar to the application above, alterations in the stimulus-response dynamics (or in the composition of the dynamically involved single components) of the cultures under study can be determined based on the measurements.

Moreover, there is a considerable need for accurate specific models, e.g., in form of representative digital copies, in order to set-up complex physiological simulations. These models are a generally necessary secondary product of the present invention. They are particularly employed in pharmacological development of drugs. There, the use of elaborated models can reduce the number of experiments with cell cultures and organisms, particularly in the early phase of development, as first insights, relationships and predictions can be drawn from simulations.

In such simulations, already performed experiments can be reperformed and reenacted; furthermore, the dynamical behavior of a specific ionic-electrical system can be predicted for certain different external or internal conditions. Especially in the case of physical models, which have at least partly a physical meaning, in opposition to pure Black-Box or regression models, the effect of certain factors of influence—e.g., genetic expression but also chemical or physical influences on certain single channels, which are involved in the ionic-electrical system, or the availability of so-called messengers or other molecules—can be estimated simulatively in case their principle of operation is at least partly known. Moreover, parameter studies can be easily performed in model simulations.

Such proceeding helps to reduce laboratory and material cost, is considerably less time consuming and eventually it is more ethical. The time savings render “mass screenings” possible, whereas a huge number (at least some thousand) of parameters, such as genetic expression of different proteins, chemical, pharmacological, toxicological, biological or physical influences or physiological messengers, is being tested. An equivalent examination featuring so many different parameters in cell cultures or organisms would not be feasible. Moreover, the combination of separate models of individual ionic-electrical systems to a joint (communicating) system allows analyzing almost arbitrary complex systems using a modular design. Their combinatorial complexity increases exponentially: e.g., analyzing a combination of n different parameters with known corresponding models of System A together with m different parameters with known corresponding models of System B yields n times m combinations. For simulating all these combinations, at most the sum of n and m models has to be known (and accordingly determined beforehand by extensive measurements); an actual conduction of all these experiments would entail a much higher number of measurements (the combinatorial combination, i.e., n times m measurements).

The present invention has the advantage that such models can be established and calibrated very saving time and cost compared to presently existing procedures. Moreover, this invention allows formulating above-mentioned models (i.e., in essence a copy of the dynamics) of ionic-electric systems, e.g., of cells, cell cultures, cell aggregates, and organisms, in such a way that the test objects are considerably less influenced or impaired and thereby they may be used for several analyses. This is possible, because—in contrast to previously known procedures, such as patch clamp—cells are not necessarily harmed or injured and single components, such as proteins etc., do not have to be isolated chemically, pharmacologically or otherwise, which influences the object under test's lifespan, functionality or just the option to reuse it without altering the results. Furthermore it is not compulsory for an analysis of the dynamics of the ionic-electrical system to specifically and irreversibly process and prepare the object under test (e.g., as tissue slices, in tissue preparation, exposure of certain cells, etc.) in order to gain access to the ionic-electric system.

Up to now, models have been created using above-mentioned procedures, e.g. patch-clamp-measurements in combination with chemical, genetical or molecular-biological isolation of components involved in the system's overall dynamics. This usually implies that overlaying other mechanisms are modified such in their behavior—usually reduced to a certain extent, slowed down or completely deactivated—for the measurements in order to analyze only a single mechanism at a time. Accordingly, parameters, for instance probabilities for certain channel states—in the simplest case the frequency ratio of open and closed state—or response times, such as state transition time constants, can be determined immediately. Such a procedure is overly complex, costly and needs an experienced operator. Therefore, only very few, scarcely specific models exist, which furthermore try to describe and mimic standard systems instead of a big variety of cases and biological components. The absence of suitable models for many ionic-electrical systems, especially in practically important physical, pharmacological, toxicological and genetic conditions limits the possibilities for the employment of simulative models in many areas, for example in pharmacological drug development.

The present invention allows ‘copying’ the ionic-electrical behavior, which hereafter will be referred to as the generation/calibration of an according computational simulation model, without the need of experimentally separation of the involved single components (e.g. their chemical or pharmacological isolation, for instance with the use of channel blockers) of the ionic-electrical system to be analyzed/copied. Instead, the individual dynamics of single components can be analyzed based on their influence on the overall dynamics by an analytical step described below.

### BRIEF DESCRIPTION OF DRAWINGS

FIG. 1 represents an overview of the present invention. An object under test (101), for example cells, cell aggregates or whole organisms becomes stimulated by a physical (for example but not limited to light, (ultra) sound, electric current, heat) or chemical (for example by addition of ion solutions, including, among others, potassium chloride, various neurotransmitters, neuro-modulators) stimulus (102) whose dynamic characteristics (for example intensity or strength over time, wavelength or frequency etc.) can be controlled. The ionic-electric response of the object under test is detected using a corresponding measuring unit and separated from disturbances by common means of filtering and signal processing (103).

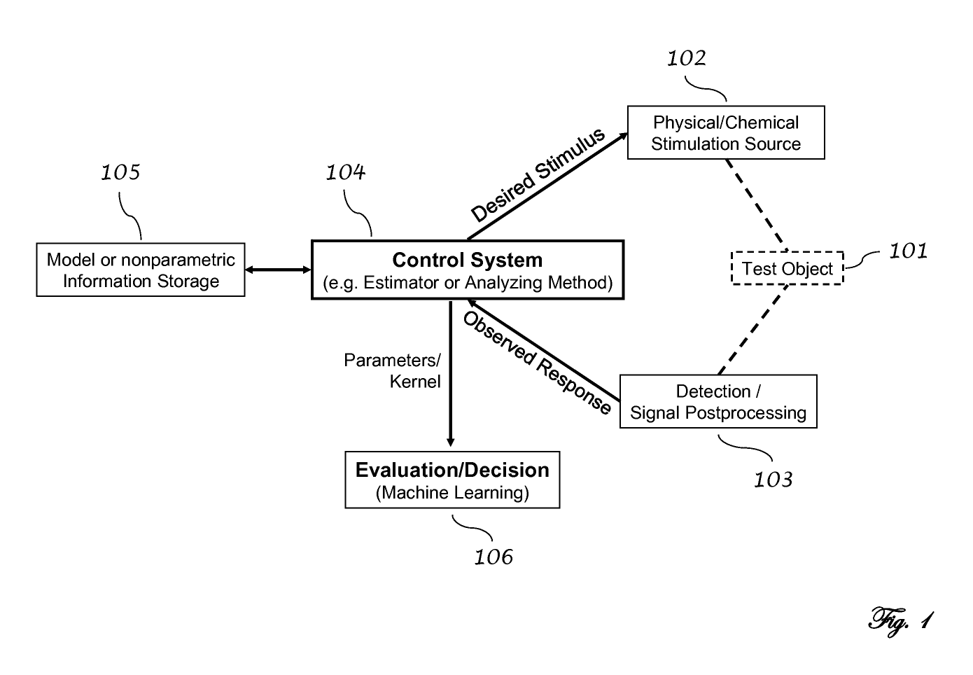

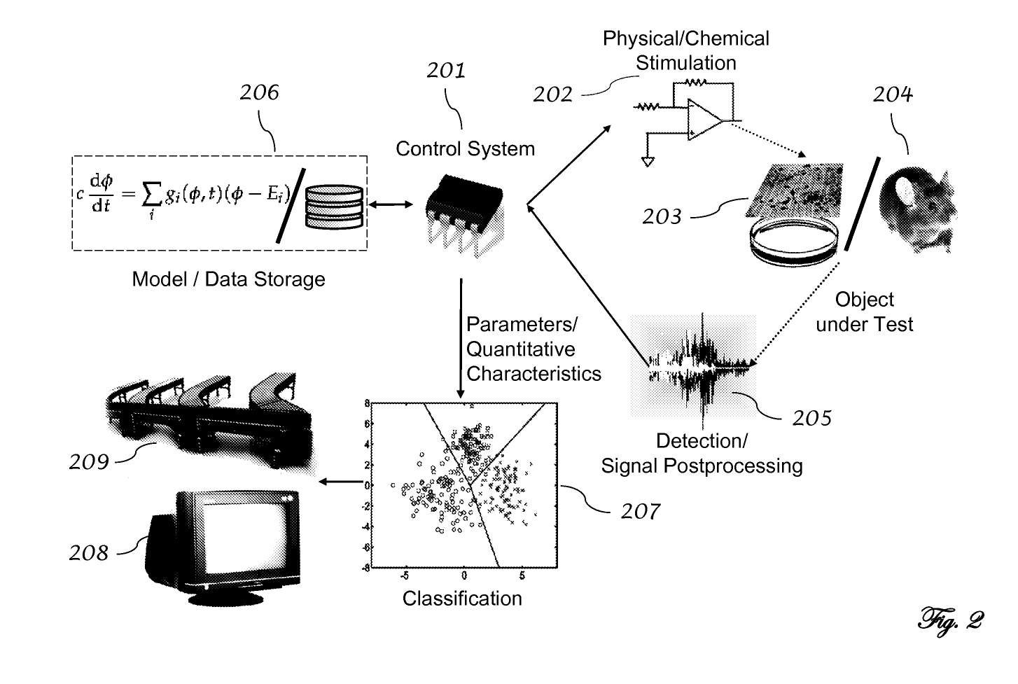

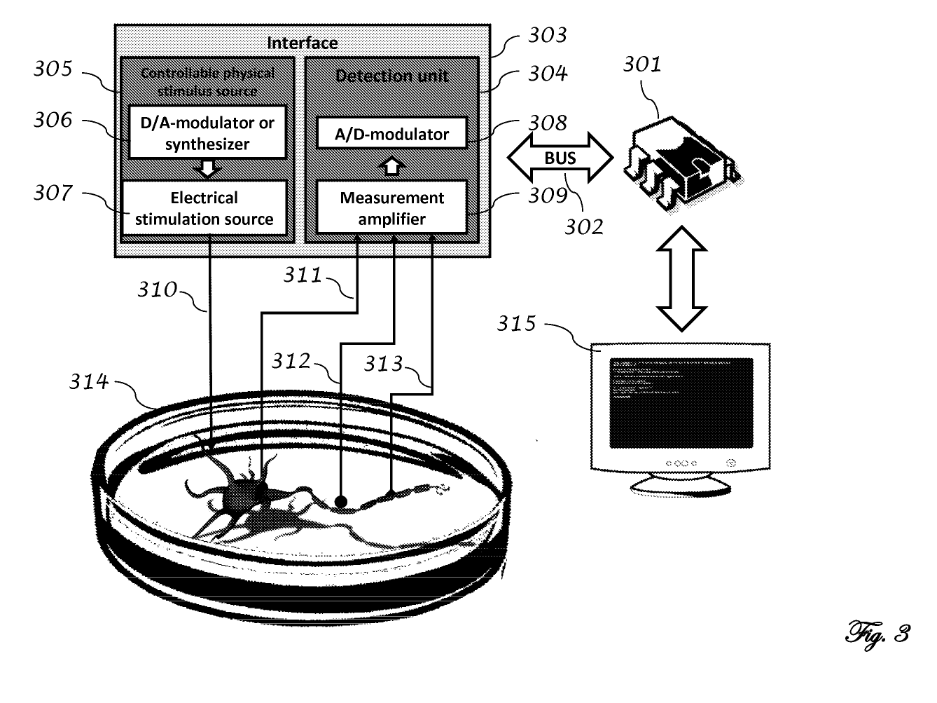

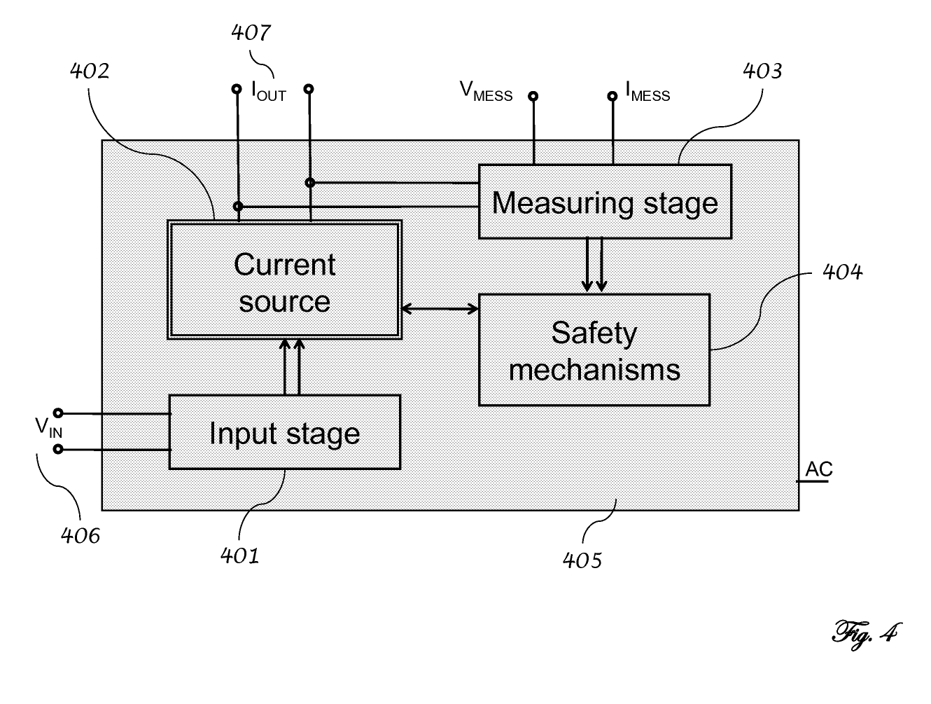

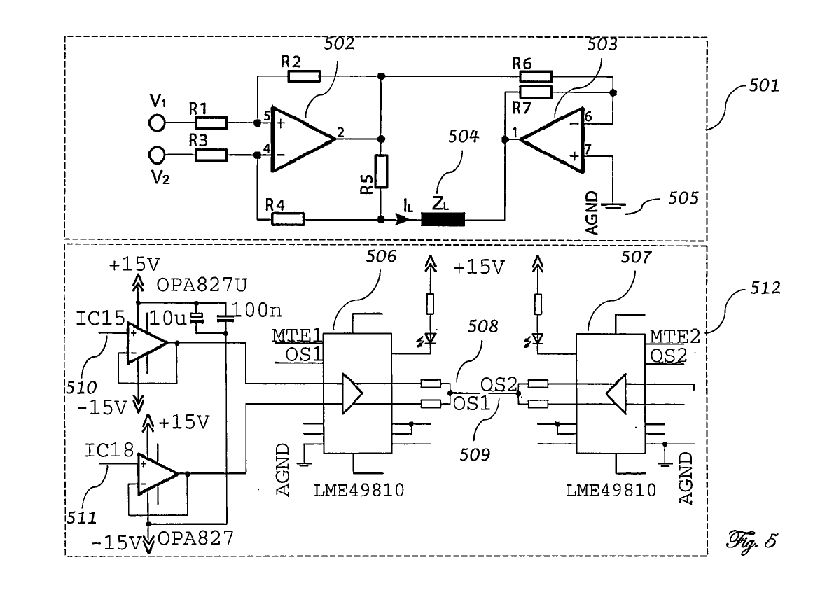

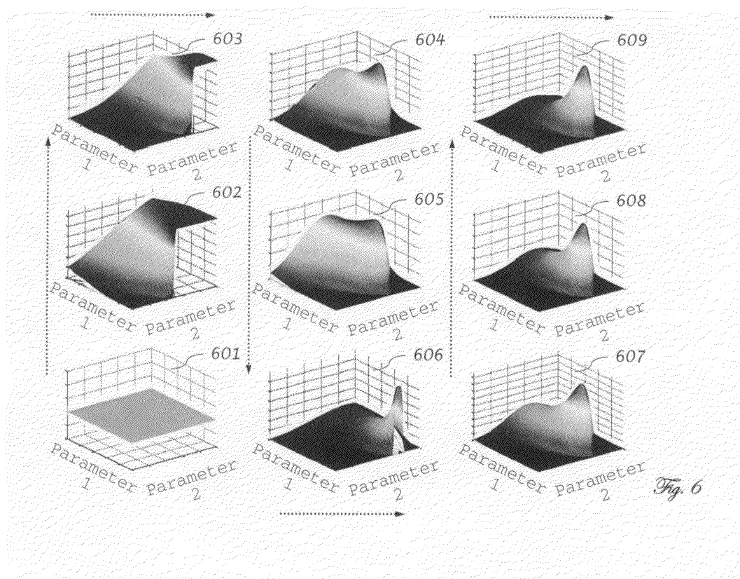

A control unit (104) analyzes the measurements and generates, based on their results, subsequent stimuli with well-defined characteristics (depending on the type of stimulus e.g. the exact time response of current intensity or voltage; alternatively the light intensity or wavelength over time or as well the added amount of a chemical over time).

The procedure uses, e.g., a mathematical-physical model of the dynamics or a nonparametric information memory for storage (105) for this task. As a result, the method and the corresponding apparatus provide a computer-implemented digital copy in terms of a parameter set or a nonparametric kernel which emulate the dynamic stimulus-response behavior of the analyzed ionic-electric system of the object under test, or lead to an abstract decision (106) to which class the analyzed ionic-electric system of the object under test belongs, for example a group that successfully expresses a subunit protein of a specific ion channel. Based on this, the object under test can be sorted accordingly.

FIG. 2 represents a preferred embodiment of the invention, where stimulation (202) and measurement (205) of the ionic-electric response occur electrically via a dynamically controllable stimulus amplifier and an electrical measurement amplifier with electrodes connected to the object under test (203, 204). Further, the analysis results are subjected to a classification (207), for example by using a principal-component analysis or a support-vector machine whose decision is displayed (208) and used for sorting and quality control (209).

The parameterized model in case of parametric analysis or the kernels in case of nonparametric analysis are provided by a corresponding unit (206), e.g. a memory. This unit stores both in case of a combined analysis with parametric and nonparametric components. Furthermore accuracy information, such as confidence intervals, probability distributions of measurement results, kernels or information from previous steps can be stored, administrated and renewed during the application.

FIG. 3 represents an particularly preferred embodiment of the invention. A control unit (301) operates, for example through an electronic bus (302), at least one physical stimulus source (305), here composed from at least one parameterized or otherwise controllable signal source (306), for example a synthesizer or a digital-to-analog modulator, and at least one electrical stimulation source (307) such as a controllable electric current or voltage source. For this electrical case, the ionic-electrical system of the object under test (314) is stimulated via stimulation electrodes (310), via detection electrodes for example in a differential arrangement or as separate test electrodes (311) and a reference electrode (312).

A grounding means (313) adjusts the electric potential of the object under test, for example via the electrolytic nutrient solution/measuring solution or a certain tissue part not belonging to the ionic-electric system under test, to prevent on the one hand potential fluctuations, capacitive charging effects of the object under test and similar interference phenomena and on the other hand the coupling of electric stimulation into the measurement, which becomes noticeable as artifacts and can affect the stability and the sensitivity of the measurement amplifier (309).

Test electrode (311) and reference electrode (312) as well as the grounding equipment (313) can be configured in different ways, usually adapted to the object under test (314): for example as conductive electrode structures on a cell culture substrate, as electrodes inserted into the cell (via established methods such as classical solid conductors or pipettes filled with electrolytic conductors as in the patch-clamp method) as needle electrodes, which detect the ambient electric potential of the cells involved in the ionic-electric system or in particular in entire mechanisms as surface electrodes which have a resistive or a capacitive contact with the organism.

The detection is performed by at least one measurement amplifier (309), whose signal is digitized over at least one analog-to-digital modulator (308) or a comparable unit. Measurement amplifier (309) and analog-to-digital modulator(s) (308) can be integrated in a detection unit (304) which is therefore responsible for the detection and signal processing of the response of an ionic-electric system of the object under test. The controllable physical stimulus source (305) and the detection unit (304) can together represent the interface (303) to the object under test (314). Results are presented to the operator, for example, on an output unit (315).

FIG. 4 shows one possible embodiment of an electrical stimulation source with current control. An input stage (401) processes and adapts the signals Vᵢₙ of a controllable signal source (406), for example a synthesizer or a digital-to-analog modulator, for the current source (402) that can contain, for example, a feedback control, and generates a stimulus current profile over time Iₒᵤₜ (407) corresponding to the input signal or linked to this signal by mathematical operations.

A measuring stage (403) determines the stimulation parameters V_{MESS} and I_{MESS}, on whose basis safety mechanisms (404) decide if critical parameters, e.g. for the object under test, are reached or exceeded, for example due to a defect, e.g. in the power supply (405), the incoming signal Vᵢₙ (406) in the current source (402) or other sources of error inside or outside the stimulation system. Therefore the current source (402) can be switched off, limited or otherwise influenced. The power supply (405) can be fed for example from the electric mains.

FIG. 5 illustrates a preferred embodiment of the current source as circuit diagram (501) and as an exemplary implementation (512) from FIG. 4 (local reference (402)) as a voltage controlled power source. The generation of the current over time through the load (504), in this case the object under test, occurs in this connection by an H-bridge configuration (501) of voltage or current sources (502) and (503), which can be implemented for example in each case as amplifier circuits. Thereby the first source (502) controls the signal shape given by the input voltages V1 and V2 using a closed-loop control and also controls the second source (503) that has no separate feed-back system. As the opposite pole to the first source (502) the second source can increase the voltage across the load Z_{L} (504) by “mirroring”, i.e., by inverting the output voltage of the first source at node 2 in its own output (node 1), in order to increase the total voltage range without changing the supply voltage or any internal device voltage as well as to improve the maximum possible rising edge speed.

The object under test and the necessary electrode material, (patch-clamp) pipettes, connecting cables etc. are summarized as complex load resistance Z_{L} (504). An exemplary implementation (512) represents the conversion of an input signal (510), (511) by two integrating amplifiers (506) and (507) into a time-current-curve through the stimulation electrodes (508) and (509). The input signal is evaluated as the potential difference between the voltages V₁ and V₂ ((510) and (511) in the exemplary implementation in (502)) which, as function of resistor-value ratio shown in (501), is converted to a current I_{L} (according to (407) from FIG. 4) through the load Z_{L}:

$${I}_{L} = \frac{{V}_{2} ( \frac{{R}_{4}}{{R}_{3}} ( \frac{{R}_{2}}{{R}_{1}} + 1 ) + \frac{{R}_{5}}{{R}_{3}} ) - {V}_{1} ( \frac{{R}_{2}}{{R}_{1}} ( \frac{{R}_{4}}{{R}_{3}} + 1 ) )}{( \frac{{R}_{5}}{{R}_{L}} ( 1 + \frac{{R}_{2}}{{R}_{1}} ) + \frac{{R}_{5} + {R}_{4}}{{R}_{3}} - \frac{{R}_{2}}{{R}_{1}} ) {R}_{L}} .$$

Provided that R₁=R₃ as well as R₂=R₄ for the current IL through the load Z_{L} is valid:

$${I}_{L} = \frac{{V}_{2} ( ( 1 + \frac{{R}_{2}}{{R}_{1}} ) + \frac{{R}_{5}}{{R}_{2}} ) - ( \frac{{R}_{2}}{{R}_{1}} + 1 ) {V}_{1}}{{R}_{5} ( 1 + \frac{{R}_{1}}{{R}_{2}} + \frac{{R}_{L}}{{R}_{2}} )}$$

Provided in addition that R₂>>R₅ and R₂>>R_{L} the relation between the input and the output signal is simplified to:

$${I}_{L} = \frac{( {V}_{2} - {V}_{2} ) \frac{{R}_{2}}{{R}_{1}}}{{R}_{5}}$$

The reference potential (505) of the current source can be adjusted regarding the object under test.

FIG. 6 shows as an example the estimated probability (z-axis) of different parameter values (x- and y-axis) of a Bayesian parameter estimator for a two dimensional model. Based on a lack of information at the beginning in step (601) (in the absence of so called prior information) a maximum of additional information is identified by systematic selection of stimuli parameters in each step, from step (601) up to step (609) in order to maximize the probability of a model parameter set that represents the analyzed object (peak in the distribution) and to reduce the probability of other parameters to a minimum (peak with a small width and absence of adjacent maxima in the probability distribution).

### DETAILED DESCRIPTION OF THE INVENTION AND EMBODIMENTS

### Analysis of the Dynamics

The dynamics of the ionic-electric system to be analyzed for may be characterized in the present invention in two ways or rather analyzed synonymous. One the one hand, a predetermined parameterized mathematical approximation of the expected dynamics may be used. Usually, these are a single or a system of (usually nonlinear) differential equations whose constants or factors are used as parameters for a calibration of the analyzed ionic-electric system.

Examples of such models are numerous in the scientific literature. Regarding a neuron, simple black box models can be used which are not physically based models [EM Izhikevich (2003). Simple model of spiking neurons. IEEE Transactions on Neural Networks, 14 (6), 1569ff]. An exemplary, but far from exhaustive list of other models can be found in [EM Izhikevich (2004)). Which model to use for cortical spiking neurons. IEEE Transactions on Neural Networks. 15 (5), 1063ff]. The advantage of not physically based models is usually the low number of parameters, which limits the flexibility and accuracy of the model regarding the real biological systems but also simplifies and accelerates the training sequence, i.e., model matching. Other examples are a nested design

$$\frac{d v}{d t} = {c}_{0} + {c}_{1} v + {c}_{2} {v}^{2} + {c}_{3} {v}^{3} + s ( t )$$

$$or$$

$$\frac{d v}{d t} = {\sum }_{i = 0}^{n} {c}_{i} {v}^{i} + s ( t ) ,$$

where v refers to the cell potential, s(t) is the stimulus, and c, denote constants, of which several can be fixed to zero or a nonzero constant before regression to keep the number of degrees of freedom low. Furthermore, this design allows a parallel regression of several models of the same kind, in which different constants cᵢ are set to fixed values, and an identification of the statistically most advantageous model. Appropriate approaches from other fields of statistics are widespread in the literature under the terms Bayesian information criterion for model identification and Akaike criterion for model identification.

In addition, physical models whose parameters and parts have a physical/chemical/biological significance, such as describing a particular protein, can be used for the analysis. Examples and nature of the dominant classes of such descriptions are illustrated in [C Koch (1999). Biophysics of Computation. Information Processing in Single Neurons. Oxford University Press, New York/Oxford.]. In this case the overall dynamics is usually divided into the individual elements involved (predominantly ionic channels) and their individual dynamic description and then combined according to the ionic-electric system or cell type of interest. For a large number of these ionic channels both genetic coding and associated dynamic description are treated in the scientific literature.

Such physical models are determined exclusively based on patch-clamp measurements with (generally chemically) isolated individual components, such as specific ion channels. As stated above, this approach differs substantially from the one in the present invention—the individual components, for example, are so far unknown—and has distinct disadvantages.

For the above example of neurons, those cells with the best known ionic-electric system, such ionic channels can be embedded along with the associated dynamic descriptions of other components(such as proteins) with a specific spatial distribution into a geometric-morphological representation of an equivalent neural cell, which accurately describes the morphology. [see, for example, C Koch (1999). Biophysics of Computation. Information Processing in Single Neurons. Oxford University Press, New York/Oxford].

Unlike to such geometric-morphological morphological models which require a multitude of parameters to be calibrated for the process described above, the dynamic analysis of ionic-electric systems moreover permits in many cases the use of mere descriptions of the stimulation dynamic.

This is also possible in a physically based model. In contrast to the above described models, such descriptions are not well established in the scientific literature. In this case, only the physical/chemical excitation is modeled, without any spatial transmission effects. For this excitation model, known descriptions of single channels and other components from the literature can be used and combined to a local, single-segment model.

The number of parameters to be calibrated decreases in such a model significantly without a considerably loss of accuracy. If the analyzed system is a neuron or a multi-cell system, the model can be supplemented with a simplified parameterized delay dynamics, such as a dead time, to represent the delay between physical/chemical stimulation and measurable electrical response, for example due to the signal transmission, in the model.

Methods for performing this calibration, also called parameter estimation, model training, or regression, are known in mathematics and applied disciplines, such as physics and statistics. For example, the parameters can be determined with a so-called maximum-likelihood approach according to R. A. Fisher, Bayesian regression, the minimization of a deviation measure such as the sum of the squared deviations (least mean squares), the median of the deviations or the maximum deviation.

Since neurons have strongly nonlinear characteristics with a tendency to chaotic behavior, parallel training of all model parameters at the same time is preferably avoided. The training can instead start with those parameters determining the dominant linear behavior (often a low-pass characteristic, rarely band pass or high-pass characteristics) whereas the remaining parameters will be set to certain known—and probable or expected values or, at least, significantly limited in their freedom of choice or allowed value range, for example, the interval. Subsequently, the remaining parameters can be estimated equivalently step by step.

In this way, increased analysis stability and speed can be achieved. In order to increase the accuracy and to correct, where necessary, existing errors in particular parameters, iterative training can be performed. Therefore, after performing an iteration for the determination of all parameters (wherein only a part of the parameters is determined in each step while the other parameters are fixed or restricted), the method can start again with already in a previous step estimated parameters so that changes of other parameters can lead to potential updates also in these already processed parameters. The sequence of parameters with respect to the previous iteration may also be varied. The iteration may be repeatedly processed, until the values stabilize.

Besides this parametric analysis method, the invention may also be used with nonparametric methods. Such methods shape the dynamics using a kind of nonlinear transfer function similar to a Taylor-series for static functions. This can be based for instance on the theoretical work of V. Volterra and N. Wiener. Below is a general representation of series r (t) of nonlinear dynamics of the form:

$$r ( t ) = {K}_{0} + {\sum }_{i = 1}^{∞} \frac{1}{i !} \int … {\int }_{{ℝ}^{i}} {K}_{i} ( {τ}_{1} , {τ}_{2} , … , {τ}_{i} ) x ( t - {τ}_{1} ) x ( t - {τ}_{2} ) … x ( t - {τ}_{i} ) d {τ}_{1} d {τ}_{2} … d {τ}_{i}$$

Thereby Kᵢ(τ₁, τ₂, . . . , τₙ) represents the so-called kernel or kernels, i.e. functions that need to be determined for the corresponding dynamic system, so that above expression describes the total nonlinear system dynamics. x(t) is the dynamic system's input signal, the counter or index i gives the order of corresponding kernel (beginning with a constant K₀ for the 0^{th} order and the linear impulse response K₁(t) to the higher orders) as well as the related sum term. For an approximate characterization of a system dynamics, the identification of a limited number of kernels will generally be sufficient.

The determination of the kernels for each nonlinear order may preferably start successively with the lowest, i.e. 0^{th} or 1^{st} one. The stimuli, as subsequently described, can be random, i.e. following a noise process or a random walk profile but may also incorporate very specific patterns or may intentionally be generated such as to lead to a fast convergence.

In contrast to the parametric analysis, which calibrates a model that does not necessarily in all cases exactly reproduce the ionic-electrical system of the object under test (see above) but can only find the closest approximation within its framework, the nonparametric analysis is usually able to describe any kind of dynamic behavior.

Both variants provide an analysis result that is at the same time both a copy of the dynamics of the ionic-electrical system and a source for computational predictions without any further characterization of the real ionic-electrical system.

Furthermore, the analysis can be performed as a combination of a model-based, parametric analysis together with a nonparametric analysis. In this case, the descriptive copy of the dynamic behavior of the ionic-electrical system is formed by both results. This can be for instance a sum or another type of mathematical operation together with a nonparametric description, e.g. a description based on the theoretical work of V. Volterra and N. Wiener. In case of a sum or another basic arithmetic method, a parametric analysis as described above can be performed, for instance, in such a way that the resulting calibrated parameters describe the dynamic behavior, i.e. the dynamics of tested ionic-electric system, as accurately and closely as possible for the limited model. The deviation between the measured behavior of the tested ionic-electric system and the calibrated model can now be analyzed using the nonparametric method. If a sum of both descriptions is used, the nonparametric analysis is accordingly applied directly on the deviation between model and measured dynamics. However any other mathematical combinations and operations can be used as well. In case a mathematical invertible operation is chosen, the whole process is particularly advantageous.

The combination of parametric, i.e. model-based analyzes with nonparametric analyzes enables using the advantages of both. These are mostly a faster, i.e. with less test stimuli, and a more stable analysis for that part which is described by the model, while the deviation that is not covered by the model can be described as accurate as desired by the nonparametric analysis so that the limitations related to the limited valid range of the parametric analysis vanish.

As a consequence, also a very simplified model with only few parameters can be used. Such a model may otherwise not be able to describe the behavior of all ionic-electrical systems under test to a sufficient accuracy but may allow in most cases a very quick calibration; the deviation can be reduced to any desired extent using the nonparametric analysis. Consequently the restrictions due to the simplified model selection do no longer exist. Also several simple models with corresponding analysis can be combined with a nonparametric analysis using arbitrary mathematical operations, including e.g. basic arithmetic operations.

The one or the several models, for example also parameters and other statistical Information in case of a parametric or hybrid analysis or the kernels describing the model in case of a nonparametric or hybrid analysis, are preferably stored in a digital memory.

In addition to the calibration, training or parameter-estimation method and the model, a module for selecting adequate and relevant physical/chemical stimuli is required for the analysis. This may in turn be performed parametrically. In this case, a stimulus is described by its quantifiable properties—e.g. amplitude, current strength, voltage, light intensity, concentration (of chemical substances) and the like—as a function of time, frequency, or wavelength in case of acoustic, mechanical or optical stimuli. In this case, the selection of the next stimulus can be performed using a sensitivity analysis.

For this purpose, those stimulus parameters are determined whose changes have the strongest influence on the calibration results respectively on their accuracy, e.g. measured as confidence interval (as typical in frequentistical statistics) using cross validation or as Bayesian probability distribution of values for every parameter in the calibration results. Such a sensitivity analysis can be implemented statistically or information-theoretically. Therefore the expected sensitivity value is, for example, calculated for every possible stimulus. As a result, that stimulus with the highest expected value is used. As an alternative, also that stimulus with the highest expected change in the entropy of the probability distribution (for example in case of Bayesian calibration methods) of the calibration results may be used as well.

As an alternative to a efficiency-based objective, i.e. commonly the most exact and accurate determination of all parameters with the lowest possible number of stimuli, also an as efficient sorting as possible, i.e. commonly a most exact and accurate classification with least stimuli, can be used as key criterion for selecting or generating the next stimulus. Therefore the selection respectively the generation of the next stimulus is connected with the following method of classification. The above mentioned sensitivity analysis is also applicable in such cases; however, the sensitivity analysis is not applied to the model parameters but to the classification and its reliability (for example their statistical measures such as confidence intervals or probabilities of misclassification, i.e. type-1 and type-2 errors, etc.).

Furthermore, the possible stimuli can be predefined so that there is no need to generate the next stimulus but that the best suitable one can be selected from the set of stored or otherwise predetermined stimuli. This is equivalent to a parametric generation if the available stimuli are parameterized in a simple way; in the most simplistic case, they are just numbered. In addition, the selection of the next stimulus can be influenced stochastically and can aim, for instance, for a certain frequency distribution of the values of certain parameters.

In case the stimulus is not parameterized but described as a precise function over time, the function over time can be parameterized on the one hand by sampling, similarly to an analog-to-digital or digital-to-analog conversion, or on the other hand by using spline curves, Fourier series, wavelets or similar procedures. The stimulus over time can be generated in a completely nonparameterized fashion by performing a sensitivity analysis for the local slope, i.e. derivative, (or higher-order derivatives where necessary) at every time step in order to obtain for a most efficient information collection process from the object to be tested the optimum or at least a very characteristic stimulus over time (given by its local slopes for every point of time).

The stimuli can be generated by the control unit before (offline) or during (online) the stimulus application.

Analysis, generation and application of the next stimulus can be performed iteratively in an alternating pattern. The stimulus generation may also be performed prior to the analysis based either on the model and its mathematical properties or can be predefined based on empirical well-suited stimuli. Moreover, based on this, the analysis can be implemented in an iterative-approximate manner, i.e. it is refined with each pair of stimulus and measured response, but may never reach the status of an exact description of the ionic-electrical system dynamics. However it can, depending on the procedures and/or model, reduce the deviation beyond any extent.

### Classification/Sorting

Based on the analysis results, a classification can be performed in the present invention either for sorting or quality control. The classification can be performed either supervised, based on known classes, usually represented by a large number of representative result parameter sets from the analysis of representative examples of the respective classes, or freely, in an unsupervised manner, i.e. after a certain number of analyses of different ionic-electrical systems or test objects this invention freely groups similar and separates distinct groups of analyzed ionic-electrical systems or object under tests that are related among each other within a group and unlike members of the other groups.

The analysis of the necessary representatives of the former, supervised case, which form the so-called training set and which determine the groups, can be performed with the method itself, in particular the analysis so that the method can become adaptive—i.e. each measurement can, together with some potential information from a supervisor, be included into its own training set—or may originate from other sources, such as the classical patch-clamp approach with chemically isolated individual components, or computer simulations.

In case of the parametric approach, the analysis provides for each analyzed ionic-electrical system or object under test a set of parameters, preferably with its associated statistical information, such as confidence intervals, parameter probability distributions etc. These results can be used in a classification to assign them to a particular group, for example the groups “correctly expressed protein A” versus “not correctly expressed protein A”, and to make a decision for the respective object under test based on this result or to sort it accordingly. Furthermore, the classification may contain more than two groups which may be complementary but not necessarily.

The classification can be based on the results of the parametric analysis, e.g. using a threshold decision, i.e. by assigning (multidimensional) intervals or ranges in which the parameter values of the members of a corresponding class lie. Furthermore a classification is possible by using an independent component analysis, also principal component analysis, of the parameters and subsequent threshold decision, soft-logic decision or statistical decision in which probabilities are assigned based on the Bayesian analysis for example. In addition, the Support Vector Machine approach can be used for a nonlinear classification. More traditional methods of machine learning such as neural networks can be used as an alternative to the ones described.

In contrast to parametric analyses, the nonparametric method, e.g. the kernel analysis on the theoretical basis of V. Volterra and N. Wiener, does not provide parametric values, i.e. a finite number of fixed values, Instead, the nonparametric analysis provides a number of functions that describe, e.g. as one or several kernels, the single linear and nonlinear dynamic orders. In general, the classification cannot be performed with known methods from the literature in this case. Instead, a similarity metric may be used in order to determine the distance of two kernel functions, e.g. between the currently analyzed ionic-electrical system and all the representatives in the training set, or between several analyzed ionic-electrical systems. Mathematical standards can be used to determine such a distance, for instance p-norms (for example, the cumulated or integrated square deviation), vector distance metrics, scalar/inner products (on finite and infinite vectors, e.g. elements of the Hilbert space L²), cross-correlation functions etc.; alternatively a statistical approach which provides as a kind of likelihood or as a Bayesian approach probabilities based on the training set, is possible as well: The kernels as functions w=kW of a corresponding class in the training set or other measurements for every order, where T is a vector and w is the function value of a kernel, which can be multidimensional, can be combined in such as to form a kind of probability density for every pair of (τ, w), accordingly therefore a (multidimensional) distribution function, i.e. a probability that a certain point (τ_{distinct}, w_{distinct}) is value of a kernel function of a particular class and order (probability P(τ_{distinct}, w_{distinct})). For this purpose, known methods for generation of probability distributions from measurement values can be used. With this probability function ƒ_{o,k}(τ, w), with a certain order o and class k, the probability that a certain kernel of a specific order is part of the specific class k can be evaluated as follows:

∫ . . . [symbol] ƒ_{o,k}=(τ₁, τ₂, . . . , τₙ),w=Kₒ(τ))·Kₒ(τ)dτ₁ . . . dτₙ

With these probabilities for each kernel order of the analyzed ionic-electrical system to belong to the particular class k, for every class the corresponding total probability, i.e. for all orders, of belonging to this class can be specified (product of the single probabilities) in order to determine not only the most probable corresponding class, but also to predict the risk of errors.

Alternatively to the probability distribution as a kind of density function of the kernel or ‘shadow’ of the kernels on the corresponding space ƒ_{o,k}(τ, w), the same method can be applied, for example, to their derivative of the kernel functions.

If the nonparametric analysis method separates the kernels of the different orders, a distance or probability comparison of several analyzed ionic-electrical systems or objects under test between each other or individually compared with the training set can be performed both separately for every order or together; therefore, the single distances or the single probabilities can be either (dependent on which of the above give metric is chosen) directly combined in a mathematically consistent way or include a potentially different weighting of all single contributions. This weighting may include, for example, a reliability metric; for example, nonlinearities and therefore kernels of higher order are generally affected by higher errors.

Also a free, unsupervised classification can be performed according to known methods from the scientific literature if accordingly the element of the distance function named here is known. The classification can be performed at the end of the analysis on the basis of the analysis results. However, if the running analysis provides results that are refined after every stimulation/excitation and corresponding response measurements, a classification can already be performed during the analysis.

### Combined Analysis and Classification

For very simple classifications, the analysis can be combined with the classification, while either the model is significantly reduced and adapted to the classification, e.g. such that it uses only the degrees of freedom of the classification itself, or such that it limits the type of estimated components in the nonparametric kernel analysis, such as the order of nonlinearities, to those nonlinearities that are most particular or characteristic for the classification or prioritizes them. This can be done, for example, by adjusting the model parameters to the dominant degrees of freedom of the classification, e.g. in terms of the number of parameters and/or the shape of the model. For example, in the ideal case the model can use exactly the dominant parameters of the classification (for example, those detected by a principal component analysis) so that the degrees of freedom of the classification are not secondary.

The extent of adjustment of the model parameters to the classification can be determined, for example, using the correlation matrix or the covariance matrix of the analysis parameters and the classification parameters. The higher the adjustment of the model to the classification, the closer the covariance matrix comes to a diagonal matrix. Furthermore the adjustment can also be performed by an appropriate choice of stimuli so that the stimuli are deliberately chosen in such a way that they enable a quick decision in the space that is given by the degrees of freedom of the classification.

### EMBODIMENTS

A preferential embodiment of the invention contains at least one above described analysis, at least one above described generation or selection of the next stimulus, at least one controllable physical/chemical stimulus source and at least one detection unit. In an especially preferred embodiment, the analysis conducts a calibration of a parametric model. In another especially preferred embodiment, the analysis is nonparametric. In a third especially preferred embodiment, a nonparametric and a parametric analysis are combined. In an especially preferred embodiment, the single elements of the above mentioned embodiments are divided into at least two separate modules that exchange electronic data with each other.

In a further preferred embodiment, all single elements, i.e. the at least one analysis, the at least one module for the generation or selection of the next stimulus, the at least one controllable physical/chemical stimulus source, the at least one detection unit, are separated into individual modules that exchange electronic data with each other.

In an especially preferred embodiment, the individual modules of the two last mentioned embodiments are also physically separated in the apparatus and possess separate electronic components that communicate in a digital or analog way with each other over electronic signal lines or bus systems (electrical, optic or other known ways of signal transmission).

Another preferred embodiment contains at least one above described analysis, at least one above described generation or selection of the next stimulus, at least one above described classification, at least one controllable physical/chemical stimulus source and at least one detection unit.

In an especially preferred embodiment, the analysis performs a calibration of a parametric model. In another especially preferred embodiment the analysis is nonparametric. In a third especially preferred embodiment, a nonparametric and a parametric analysis are combined. In an especially preferred embodiment, the single elements of the above mentioned embodiments are divided into at least two separate modules who exchange electronic data with each other.

In a further preferred embodiment, all single elements, i.e. analysis, next stimulus generation or selection, controllable physical/chemical stimulus source, detection unit, are divided into individual modules that exchange electronic data with each other.

In an especially preferred embodiment, the individual modules of the two last mentioned embodiments are also physically separated in the apparatus and use separate electronic components which communicate digitally or analogously with each other via electronic signal lines or bus systems (electrical, optic or other signal transmission way).

Another preferred embodiment contains at least one above described analysis, at least one above described generation or selection of the next stimulus, at least one above described classification, at least one controllable physical/chemical stimulus source and at least one detection unit.

In another particular embodiment the entire process consisting of at least two of the following steps runs iteratively: application of a stimulus, response detection, analysis of this single stimulus-response pair, analysis of all previous stimulus-response pairs and generation of a new stimulus based on a statistical sensitivity analysis or from a predefined data base.

Claimed is:

1. A device for generating digital copies of at least one biological cells under test such that the digital copy imitates the dynamical ionic-electrical behavior of the at least one cells under test regarding distinct ambient condition parameters, and compromising:
a stimulus source to perform one or more stimuli to the at least one cells under test, wherein a stimulus is a biological, chemical, pharmacological or physical manipulation of the at least one cells under test with limited duration in such a way that the ionic-electrical behavior of the at least one cells under test is changed at least briefly and
a detection unit to measure and store the stimulus-response of the at least one cells under test, wherein at least one physical quantity of the at least one cells under test that have been influenced by the one or more stimuli and the ambient condition parameters are measured, and
a control unit to generate a dynamical model of the at least one cells under test as a digital copy of the at least one cells under test in such a way, that the model imitates its/their response to a stimulus well.

2. A device according to claim 1, wherein the device comprises means to generate and/or to influence and/or to measure the biological, chemical, pharmacological and/or physical ambient condition parameters of the at least one cells under test.

3. A device according to claim 2, wherein the ambient condition parameters are among the following biological, chemical, pharmacological and/or physical conditions, that influence the ionic-electrical behavior of the one or more cells and which change slowly compared to the duration of a stimulus:
temperature;
air pressure;
mechanical pressure;
humidity;
electromagnetic radiation;
concentration of chemical or pharmacological substances in the medium surrounding at least one cells under test;
ion concentration in the at least one cells under test as well as in the cell's or cells' surrounding medium;
nutrient concentration in the medium surrounding the at least one cells under test.

4. A device according to claim 1, wherein the duration-limited stimulation by the stimulus source is performed by one of the following means or by a combination of the following means:
an optical wave;
an electromagnetic wave comprising at least one wavelength above or below the optical spectrum;
an electric field or an electric current having a predetermined spatial and temporal profile;
by the effective contact with at least one biological, chemical or pharmacological agents;
an acoustic wave;
a mechanical interaction.

5. A device according to claim 1, wherein the measurement unit measures at least one of the following quantities:
an electrical stimulus response that includes an at least temporary change in the electrical voltage conditions in the at least one cells under test or its environment;
an optical stimulus response that comprises an at least temporarily change in the optical properties of the one cell or cells or their environment;
an optical stimulus-response that includes an at least temporary emission of an optical wave by the at least one cells under test;
a mechanical stimulus response.

6. A device according to claim 5, wherein the mechanical stimulus response compromises the contraction or expansion of the at least one cells under test or of a subunit of the at least one cells under test.

7. A device according to claim 5, wherein the electrical stimulus-response is detected using an electrical amplifier as part of the measurement unit.

8. A device according to claim 1, wherein the control unit includes a predefined model for the generation of the digital copies that describes the corresponding response of the at least one cells under test to a stimulus using a finite number of parameters.

9. A device according to claim 1, wherein the control unit includes a nonparametric description for the generation of the digital copy, characterized such that the data set describing the corresponding response of the at least one cells under test to a stimulus, can be expanded to a freely chosen extent.

10. A device according to claim 1, wherein it is able to assign the at least one cells under test to a group of cells with similar behavior by comparing the stimulus response of the at least one cells under test and/or the digital copy of the at least one cells under test or data sets representing cells with corresponding data of existing digital copies.

11. A device according to claim 10, wherein the selection of groups of the one or more cells under test is made depending on cell type or on the presence and functionality of particular proteins or types of proteins in the at least one cells under test or on biological, chemical or physical ambient condition parameters of the real cells.

12. A device according to claim 11, wherein the statistical assignment unit includes a means of sorting that performs a labeling and/or physical, chemical, biological or mechanical sorting of the at least one cells under test or of the at least one cells under test together with the object, they are mechanically embedded into or mechanically connected with.

13. A device according to claim 1, wherein the at least one cells under test are measured in vivo.

14. A method for generating digital copies of at least one biological cells under test such that the digital copy imitates the dynamical ionic-electrical behavior of the at least one cells under test regarding distinct ambient condition parameters, and compromising the following steps:
stimulation—to perform one or more stimuli to the at least one cells under test, wherein a stimulus is a biological, chemical, pharmacological or physical manipulation of the at least one cells under test with limited duration in such a way that the ionic-electrical behavior of the at least one cells under test is changed at least briefly and
detection—to measure and store the stimulus-response of the at least one cells under test, wherein at least one physical quantity of the at least one cells under test that have been influenced by the one or more stimuli and the ambient condition parameters are measured, and
analysis/control—to generate a mathematical, dynamical model of the at least one cells under test as a digital copy of the at least one cells under test in such a way, that the model imitates its/their response to a stimulus well.

15. A method according to claim 14, wherein the duration-limited stimulation by the stimulus source is performed by one of the following means or by a combination of the following means:
an optical wave;
an electromagnetic wave comprising at least one wavelength above or below the optical spectrum;
an electric field or an electric current having a predetermined spatial and temporal profile;
by the effective contact with at least one biological, chemical or pharmacological agents;
an acoustic wave;
a mechanical interaction.

16. A method according to claim 14, wherein at least one of the following quantities are measured in the measurement step:
an electrical stimulus response that includes an at least temporary change in the electrical voltage conditions in the at least one cells under test or its environment;
an optical stimulus response that comprises an at least temporarily change in the optical properties of the one cell or cells or their environment;
an optical stimulus-response that includes an at least temporary emission of an optical wave by the at least one cells under test;
a mechanical stimulus response.

17. A method according to claim 14, wherein a predefined model is used for the generation of the digital copies that describes the corresponding response of the at least one cells under test to a stimulus using a finite number of parameters.

18. A method according to claim 14, wherein the generation of the digital copy is based on nonparametric description, characterized such that the data set describing the corresponding response of the at least one cells under test to a stimulus, can be expanded to a freely chosen extent.

19. A method according to claim 14, wherein it is able to assign the at least one cells under test to a group of cells with similar behavior by comparing the stimulus response of the at least one cells under test and/or the digital copy of the at least one cells under test or data sets representing cells with corresponding data of existing digital copies.

20. A method according to claim 19, wherein the selection of groups of the one or more cells under test is made depending on cell type or on the presence and functionality of particular proteins or types of proteins in the at least one cells under test or on biological, chemical or physical ambient condition parameters of the real cells.
